(19) United States
(12) Patent Application Publication
(10) Pub. No.: US 2008/0306145 A1
(43) Pub. Date: Dec. 11, 2008

(54) Preparation Of Ginkgolide And F-Seco-Ginkgolide Lactols

(75) Inventors: Koji Nakanishi, New York, NY (US); Nina Berova, New York, NY (US); Katsunori Tanaka, Osaka (JP)

Correspondence Address: COOPER & DUNHAM, LLP, 1185 AVENUE OF THE AMERICAS, NEW YORK, NY, 10036, US

(73) Assignee: The Trustees of Columbia University in the City of New York, New York, NY (US)

(21) Appl. No.: 11/791,422

(22) PCT Filed: Nov. 23, 2005
(86) PCT No.: PCT/US2005/042647

§ 371 Date: Jun. 23, 2008

Related U.S. Application Data
(60) Provisional application No. 60/631,048, filed on Nov. 23, 2004.

(51) Int. Cl. A61K 31/343; C07D 307/77; A61P 7/00

(52) U.S. Cl. 514/468; 549/297

(57) ABSTRACT

The present invention relates to synthesis of C₁₁ natural ginkgolide derivatives and C₁₁ f-seco-gingkolide derivatives from the corresponding lactols which are selectively obtained using NaBH₄.

This application claims the benefit of U.S. Provisional Application No. 60/631,048, filed Nov. 23, 2004, the contents of which are hereby incorporated by reference into the subject application.

The invention disclosed herein was made with Government support under grant nos. MH 068817 and GM 34509 from the National Institutes of Health. Accordingly, the U.S. Government has certain rights in this invention.

Throughout this application, various publications are referenced by number in parentheses. The full citation for these publications can be found at the end of the specification. The disclosures of these publications in their entireties are hereby incorporated by reference into this application in order to more fully describe the state of the art as known to those skilled therein as of the date of the invention described and claimed herein.

### BACKGROUND OF THE INVENTION

Ginkgolides from the Ginkgo biloba tree are diterpenes with a rigid cage structure consisting of six five-membered rings and a unique t-Bu groups (FIG. 1) (1). Ginkgolides exhibit a variety of biological properties, one of the earliest recognized being their antagonist properties against the platelet activating factor receptor (PAFR) (2 and 3). Recently, it has been shown that they are potent and selective antagonists of the inhibitory glycine and GABA_{A} receptors (4-6). In view of such attractive biological activities, a variety of ginkgolide analogs have been prepared (7-19). So far, however, the preparation of ginkgolide derivatives has been restricted to the functionalization of hydroxyl groups, i.e., selective acylation or alkylation of one of the three hydroxyls in ginkgolide C (18).

Another attractive approach is the modification and deep-seated transformation of the ginkgolide cage skeleton. The extensive degradation studies of native ginkgolides performed during the course of structural determination (20-26) gave rise to dilactone derivative 1 lacking the ring F of original ginkgolides (see structure in FIG. 2). It was obtained readily from ginkgolide C through methylation, acetylation, and hydrogenation. However, since the derivatization of 1 had not been explored, the current studies were performed in view of its attractive truncated skeleton as a new template for preparation of a new series of derivatives.

Here, a new series of unique ginkgolide derivatives is disclosed. The surprising and unique reactivity of the ginkgolide α-protected hydroxy lactones toward the mild and common reducing reagent NaBH₄ selectively provides unique lactol derivatives.

### SUMMARY OF THE INVENTION

One embodiment of the invention disclosed here provides a compound having the structure:

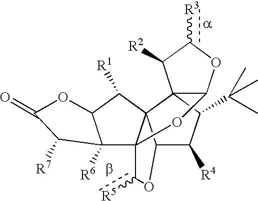

wherein R¹ is H or —OR⁸,
where R⁸ is H, or —C(O)R⁹, where R⁹ is alkyl, aryl, or amino;
   wherein R² is H, OH, —O(CH₃), —OC(O)CH₃, (C₁-C₁₀) alkyl, (C₂-C₁₀) alkenyl, (C₂-C₁₀) alkynyl, -A-Ar, -A-Z-Ar, —SO₂—Ar, -A-NR¹⁰, —O-A-Ar, or —R¹⁰,
where A is (C₁-C₈) alkyl, (C₂-C₈) alkenyl, (C₂-C₈) alkynyl, which is unsubstituted or substituted by a straight or branched alkyl chain group having 1 to 5 carbon atoms; Z is carbon, oxygen, sulfur or nitrogen; Ar is a phenyl group, a pyridyl group, a naphthyl group, a pyrimidyl group, or a quinolyl group, each of which is unsubstituted or substituted by one to five substituents selected from the group consisting of hydrogen, halogen, a hydroxy group, a carboxylic acid group, substituted or unsubstituted (C₁-C₁₀) alkyl, (C₂-C₁₀) alkenyl, (C₂-C₁₀) alkynyl, (C₁-C₁₀) haloalkyl, (C₁-C₁₀) alkoxy, (C₂-C₁₀) alkenyloxy, (C₂-C₁₀) alkynyloxy, (C₁-C₁₀) haloalkoxy, a phenyl group, a phenoxy group, an aralkyl group, an aralkyloxy group, a substituted phenyl group, a substituted phenoxy group, a substituted aralkyl group, a substituted aralkyloxy group, —C(O)R¹⁰, —C(O)NR¹⁰R¹⁰, —C(O)OR¹⁰, —N(H)COR¹⁰, —NH(OH), —N(OH)COR¹⁰, —CH₂OR¹⁰, —OCH₂CO₂R¹⁰, —CH₂CO₂R¹⁰, —CH₂SR¹⁰, —CH₂NR¹⁰R¹⁰, —CH₂CONR¹⁰R¹⁰, —SR¹⁰, —OSR¹⁰, —N(R¹⁰)(R¹⁰), or —NR¹⁰SO₂R¹⁰,
   where each R¹⁰ is independently selected from hydrogen, (C₁-C₁₀) alkyl, (C₃-C₁₀) cycloalkyl, —SCX₃ in which X is a halogen, —CN, —NO₂ or -Z-A-Z′- in which Z and A are as defined above and Z′ represents carbon, oxygen, sulfur, or nitrogen;
      wherein R⁴ is H, OH, halide, unsubstituted or substituted, straight or branched (C₁-C₅) alkyl group, (C₂-C₅) alkenyl, or a (C₂-C₅) alkynyl, (C₁-C₅) alkoxy, (C₂-C₅) alkenyloxy, or (C₂-C₅) alkynyloxy, —N₃, —C(O)R¹¹, —C(O)NR¹¹R¹², —C(O)OR¹¹, —OC(O)R¹¹, —OC(O)OR¹¹, —NH(OH), —N(R¹¹)(R¹²), —N(H)COR¹¹, —N(OH)COR¹¹, —CH₂OR¹¹, —OCH₂CO₂R¹¹, —CH₂SR¹¹, —CH₂N(R¹¹)(R¹²), —SR¹¹, —OSR¹¹, —N(R¹¹) SO₂R¹², —OR¹³ or triethylsiloxy,
where R¹³ is H, —C(O)—O—R¹⁴, or —C(O)(R¹⁴), where R¹⁴ is alkyl, aryl, or amino, and where R¹¹ and R¹² are each, independently, hydrogen, substituted or unsubstituted (C₁-C₅) alkyl, (C₂-C₅) alkenyl, (C₂-C₅) alkynyl, or cycloalkyl or aryl group having 3 to 10 carbon atoms;
   wherein R⁶ is H or —OR⁸,
where R⁸ is H, or —C(O)R⁹, where R⁹ is alkyl, aryl, or amino;
   wherein R⁷ is —CH₃;
   wherein R³ is O and R⁵ is selected from H, OH, —C(CH₃)—C(O)—O(CH₃), (C₁-C₁₀) alkyl, (C₂-C₁₀) alkenyl, (C₂-C₁₀) alkynyl, substituted or unsubstituted aryl, substituted or unsubstituted heteroaryl, heterocyclic, amino, amido, alkoxy, alkenyloxy, alkynyloxy, —O-aryl, —O-(alkyl)(C₁-C₁₀), (—NH-alkyl, —N(alkyl)₂, —NH₂, -alkyl-C(O)(OH), -alkyl-OH, -alkyl-(NH₂), halide, CX₃ where X is a halide, indole radical, -A-Ar, -A-Z-Ar, —SO₂—Ar, -A-NR¹⁰, —O-A-Ar, or —R¹⁰, where A, Z, Ar and R¹⁰ are defined as above, or
   wherein R⁵ is O and R³ is selected from OH, —C(CH₃)—C(O)—O(CH₃), (C₁-C₁₀) alkyl, (C₂-C₁₀) alkenyl, (C₂-C₁₀) alkynyl, substituted or unsubstituted aryl, substituted or unsubstituted heteroaryl, heterocyclic, amino, amido, alkoxy, alkenyloxy, alkynyloxy, —O-aryl, —O—(C₂-C₁₀ alkyl) (C₁-C₁₀), (—NH-alkyl, —N(alkyl)₂, —NH₂, -alkyl-C(O) (OH), -alkyl-OH, -alkyl-(NH₂), halide, CX₃ where X is a halide, indole radical, -A-Ar, -A-Z-Ar, —SO₂—Ar, -A-NR¹⁰, —O-A-Ar, or —R¹⁰, where A, Z, Ar and R¹⁰ are defined as above,
   or an optically pure enantiomer, diastereomer, tautomer or salt thereof.

### BRIEF DESCRIPTION OF THE FIGURES

FIG. 1: Structure of five ginkgolides.

FIG. 2: Reduction of alpha-hydroxy lactones to lactols.

FIG. 3: Reduction ratio of alpha-hydroxy lactones. Each reaction was performed using 1 equivalent of NaBH₄ at room temperature for 5 minutes. The reaction mixtures were directly acylated by p-phenylbenzoic acid and the products were analyzed by ¹H NMR. None of the over-reduced diols were observed.

FIG. 4: Synthesis of ginkgolide B lactol derivative. The C₁₁ hydroxy position provided by this synthesis can be readily broadly derivatized.

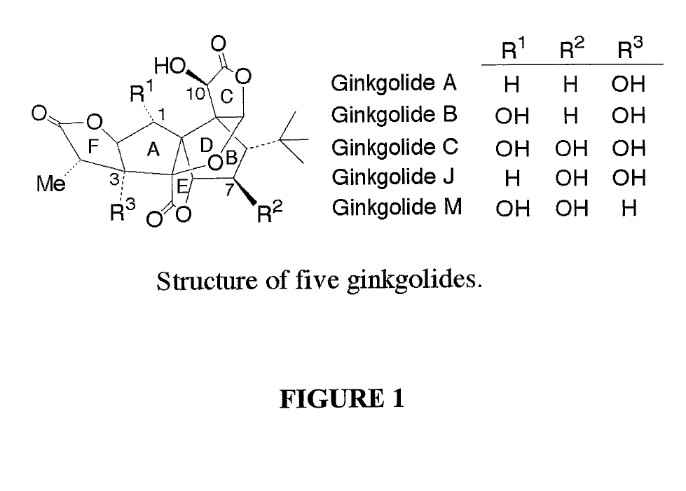

### DETAILED DESCRIPTION

This invention provides a compound having the structure:

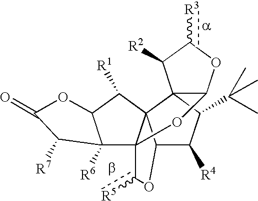

wherein R¹ is H or —OR⁸,
where R⁸ is H, or —C(O)R⁹, where R⁹ is alkyl, aryl, or amino;
   wherein R² is H, OH, —O(CH₃), —OC(O)CH₃, (C₁-C₁₀) alkyl, (C₂-C₁₀) alkenyl, (C₂-C₁₀) alkynyl, -A-Ar, -A-Z-Ar, —SO₂—Ar, -A-NR¹⁰, —O-A-Ar, or —R¹⁰,
where A is (C₁-C₈) alkyl, (C₂-C₈) alkenyl, (C₂-C₈) alkynyl, which is unsubstituted or substituted by a straight or branched alkyl chain group having 1 to 5 carbon atoms; Z is carbon, oxygen, sulfur or nitrogen; Ar is a phenyl group, a pyridyl group, a naphthyl group, a pyrimidyl group, or a quinolyl group, each of which is unsubstituted or substituted by one to five substituents selected from the group consisting of hydrogen, halogen, a hydroxy group, a carboxylic acid group, substituted or unsubstituted (C₁-C₁₀) alkyl, (C₂-C₁₀) alkenyl, (C₂-C₁₀) alkynyl, (C₁-C₁₀) haloalkyl, (C₁-C₁₀) alkoxy, (C₂-C₁₀) alkenyloxy, (C₂-C₁₀) alkynyloxy, (C₁-C₁₀) haloalkoxy, a phenyl group, a phenoxy group, an aralkyl group, an aralkyloxy group, a substituted phenyl group, a substituted phenoxy group, a substituted aralkyl group, a substituted aralkyloxy group, —C(O)R¹⁰, —C(O)NR¹⁰R¹⁰, —C(O)OR¹⁰, —N(H)COR¹⁰, —NH(OH), —N(OH)COR¹⁰, —CH₂OR¹⁰, —OCH₂CO₂R¹⁰, —CH₂CO₂R¹⁰, —CH₂SR¹⁰, —CH₂NR¹⁰R¹⁰, —CH₂CONR¹⁰R¹⁰, —SR¹⁰, —OSR¹⁰, —N(R¹⁰)(R¹⁰), or —NR¹⁰SO₂R¹⁰,
   where each R¹⁰ is independently selected from hydrogen, (C₁-C₁₀) alkyl, (C₃-C₁₀) cycloalkyl, —SCX₃ in which X is a halogen, —CN, —NO₂ or -Z-A-Z′- in which Z and A are as defined above and Z′ represents carbon, oxygen, sulfur, or nitrogen;
      wherein R⁴ is H, OH, halide, unsubstituted or substituted, straight or branched (C₁-C₅) alkyl group, (C₂-C₅) alkenyl, or a (C₂-C₅) alkynyl, (C₁-C₅) alkoxy, (C₂-C₅) alkenyloxy, or (C₂-C₅) alkynyloxy, —N₃, —C(O)R¹¹, —C(O)NR¹¹R¹², —C(O)OR¹¹, —OC(O)R¹¹, —OC(O)OR¹¹, —NH(OH), —N(R¹¹)(R¹²), —N(H)COR¹¹, —N(OH)COR¹¹, —CH₂OR¹¹, —OCH₂CO₂R¹¹, —CH₂SR¹¹, —CH₂N(R¹¹)(R¹²), —SR¹¹, —OSR¹¹, —N(R¹¹)SO₂R¹², —OR¹³, or triethylsiloxy,
where R¹³ is H, —C(O)—O—R¹⁴, or —C(O)(R¹⁴), where R¹⁴ is alkyl, aryl, or amino, and where R¹¹ and R¹² are each, independently, hydrogen, substituted or unsubstituted (C₁-C₅) alkyl, (C₂-C₅) alkenyl, (C₂-C₅) alkynyl, or cycloalkyl or aryl group having 3 to 10 carbon atoms;
   wherein R⁶ is H or —OR⁸,
where R⁸ is H, or —C(O)R⁹, where R⁹ is alkyl, aryl, or amino;
   wherein R⁷ is —CH₃;
   wherein R³ is O and R⁵ is selected from H, OH, —C(CH₃)—C(O)—O(CH₃), (C₁-C₁₀) alkyl, (C₂-C₁₀) alkenyl, (C₂-C₁₀) alkynyl, substituted or unsubstituted aryl, substituted or unsubstituted heteroaryl, heterocyclic, amino, amido, alkoxy, alkenyloxy, alkynyloxy, —O-aryl, —O-(alkyl)(C₁-C₁₀), (—NH-alkyl, —N(alkyl)₂, —NH₂, -alkyl-C(O)(OH), -alkyl-OH, -alkyl-(NH₂), halide, CX₃ where X is a halide, indole radical, -A-Ar, -A-Z-Ar, —SO₂—Ar, -A-NR¹⁰, —O-A-Ar, or —R¹⁰, where A, Z, Ar and R¹⁰ are defined as above, or
   wherein R⁵ is O and R³ is selected from OH, —C(CH₃)—C(O)—O(CH₃), (C₁-C₁₀) alkyl, (C₂-C₁₀) alkenyl, (C₂-C₁₀) alkynyl, substituted or unsubstituted aryl, substituted or unsubstituted heteroaryl, heterocyclic, amino, amido, alkoxy, alkenyloxy, alkynyloxy, —O-aryl, —O—(C₂-C₁₀ alkyl) (C₁-C₁₀), (—NH-alkyl, —N(alkyl)₂, —NH₂, -alkyl-C(O) (OH), -alkyl-OH, -alkyl-(NH₂), halide, CX₃ where X is a halide, indole radical, -A-Ar, -A-Z-Ar, —SO₂—Ar, -A-NR¹⁰, —O-A-Ar, or —R¹⁰, where A, Z, Ar and R¹⁰ are defined as above,
   or an optically pure enantiomer, diastereomer, tautomer or salt thereof.

This invention further provides the instant compound having the structure:

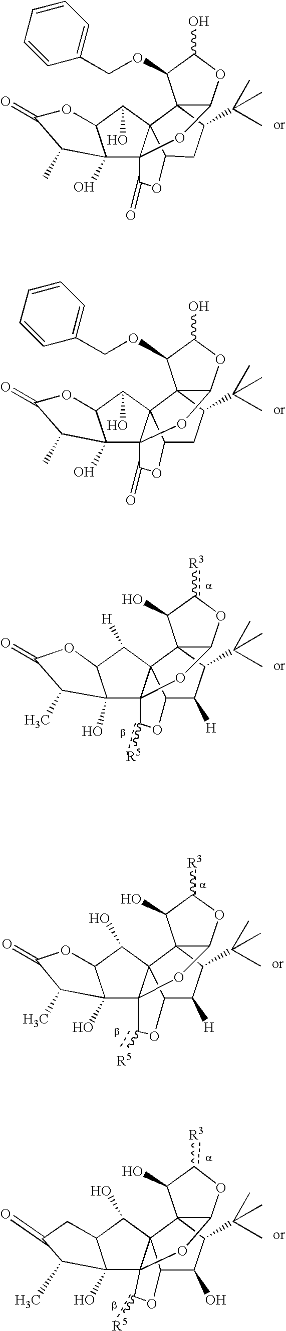

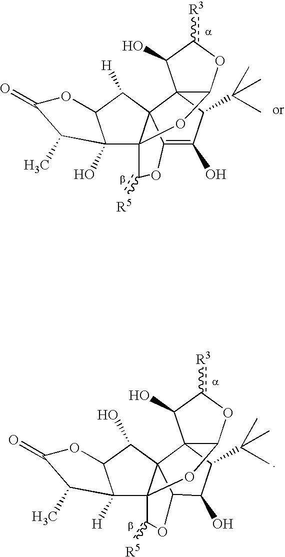

This invention also provides a process for preparing the instant compound comprising:

(a) exposing a compound having the structure:

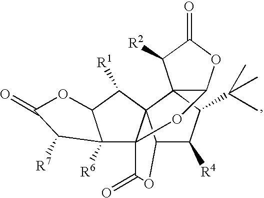

wherein R¹ is H or OR⁸,
where R⁸ is H, or —C(O)R⁹, where R⁹ is alkyl, aryl, or amino;
   wherein R² is H, OH, —O(CH₃), —OC(O)CH₃, (C₁-C₁₀) alkyl, (C₂-C₁₀) alkenyl, (C₂-C₁₀) alkynyl, -A-Ar, -A-Z-Ar, —SO₂—Ar, -A-NR¹⁰, —O-A-Ar, or —R¹⁰,
where A is (C₁-C₈) alkyl, (C₂-C₈) alkenyl, (C₂-C₈) alkynyl, which is unsubstituted or substituted by a straight or branched alkyl chain group having 1 to 5 carbon atoms; Z is carbon, oxygen, sulfur or nitrogen; Ar is a phenyl group, a pyridyl group, a naphthyl group, a pyrimidyl group, or a quinolyl group, each of which is unsubstituted or substituted by one to five substituents selected from the group consisting of hydrogen, halogen, a hydroxy group, a carboxylic acid group, substituted or unsubstituted (C₁-C₁₀) alkyl, (C₂-C₁₀) alkenyl, (C₂-C₁₀) alkynyl, (C₁-C₁₀) haloalkyl, (C₁-C₁₀) alkoxy, (C₂-C₁₀) alkenyloxy, (C₂-C₁₀) alkynyloxy, (C₁-C₁₀) haloalkoxy, a phenyl group, a phenoxy group, an aralkyl group, an aralkyloxy group, a substituted phenyl group, a substituted phenoxy group, a substituted aralkyl group, a substituted aralkyloxy group, —C(O)R¹⁰, —C(O)NR¹⁰R¹⁰, —C(O)OR¹⁰, —N(H)COR¹⁰, —NH(OH), —N(OH)COR¹⁰, —CH₂OR¹⁰, —OCH₂CO₂R¹⁰, —CH₂CO₂R¹⁰, —CH₂SR¹⁰, —CH₂NR¹⁰R¹⁰, —CH₂CONR¹⁰R¹⁰, —SR¹⁰, —OSR¹⁰, —N(R¹⁰)(R¹⁰), or —NR¹⁰SO₂R¹⁰,
   where each R¹⁰ is independently selected from hydrogen, (C₁-C₁₀) alkyl, (C₃-C₁₀) cycloalkyl, —SCX₃ in which X is a halogen, —CN, —NO₂ or -Z-A-Z′- in which Z and A are as defined above and Z′ represents carbon, oxygen, sulfur, or nitrogen;
      wherein R⁴ is H, OH, halide, unsubstituted or substituted, straight or branched (C₁-C₅) alkyl group, (C₂-C₅) alkenyl, or a (C₂-C₅) alkynyl, (C₁-C₅) alkoxy, (C₂-C₅) alkenyloxy, or (C₂-C₅) alkynyloxy, —N₃, —C(O)R¹¹, —C(O)NR¹¹R¹², —C(O)OR¹¹, —OC(O)R¹¹, —OC(O)OR¹¹, —NH(OH), —N(R¹¹)(R¹²), —N(H)COR¹¹, —N(OH)COR¹¹, —CH₂OR¹¹, —OCH₂CO₂R¹¹, —CH₂SR¹¹, —CH₂N(R¹¹)(R¹²), —SR¹¹, —OSR¹¹, —N(R¹¹)SO₂R¹², —OR¹³, or triethylsiloxy,
where R¹³ is H, —C(O)—O—R¹⁴, or —C(O)(R¹⁴), where R¹⁴ is alkyl, aryl, or amino, and where R¹¹ and R¹² are each, independently, hydrogen, substituted or unsubstituted (C₁-C₅) alkyl, (C₂-C₅) alkenyl, or (C₂-C₅) alkynyl, or a cycloalkyl or aryl group having 3 to 10 carbon atoms;
   wherein R⁶ is H or —OR⁸,
where R⁸ is H, or —C(O)R⁹, where R⁹ is alkyl, aryl, or amino; and
   wherein R⁷ is —CH₃,
   to NaBH₄ in a suitable solvent to produce a lactol derivative; and
   (b) reacting the lactol derivative product of step (a) with an agent suitable to produce the compound.

This invention further provides the instant process, wherein the suitable solvent in step (a) is MeOH.

This invention further provides the instant process, wherein step (a) is performed at room temperature.

This invention further provides the instant process, wherein the suitable agent is a carboxcylic acid, an alkylating reagent or an acid halide. In one embodiment the alkylating reagent is an alkyliodide. In one embodiment the alkylating reagent is methyliodide.

This invention also provides a process for preparing the instant compound comprising:

(a) exposing a compound having the structure:

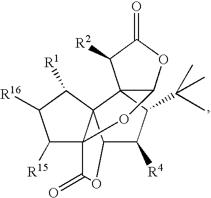

wherein R¹ is H or —OR⁸,
wherein R² is H, OH, —O(CH₃), —OC(O)CH₃, (C₁-C₁₀) alkyl, (C₂-C₁₀) alkenyl, (C₂-C₁₀) alkynyl, -A-Ar, -A-Z-Ar, —SO₂—Ar, -A-NR¹⁰, —O-A-Ar, or —R¹⁰,
where A is (C₁-C₈) alkyl, (C₂-C₈) alkenyl, (C₂-C₈) alkynyl, which is unsubstituted or substituted by a straight or branched alkyl chain group having 1 to 5 carbon atoms; Z is carbon, oxygen, sulfur or nitrogen; Ar is a phenyl group, a pyridyl group, a naphthyl group, a pyrimidyl group, or a quinolyl group, each of which is unsubstituted or substituted by one to five substituents selected from the group consisting of hydrogen, halogen, a hydroxy group, a carboxylic acid group, substituted or unsubstituted (C₁-C₁₀) alkyl, (C₂-C₁₀) alkenyl, (C₂-C₁₀) alkynyl, (C₁-C₁₀) haloalkyl, (C₁-C₁₀) alkoxy, (C₂-C₁₀) alkenyloxy, (C₂-C₁₀) alkynyloxy, (C₁-C₁₀) haloalkoxy, a phenyl group, a phenoxy group, an aralkyl group, an aralkyloxy group, a substituted phenyl group, a substituted phenoxy group, a substituted aralkyl group, a substituted aralkyloxy group, —C(O)R¹⁰, —C(O)NR¹⁰R¹⁰, —C(O)OR¹⁰, —N(H)COR¹⁰, —NH(OH), —N(OH)COR¹⁰, —CH₂OR¹⁰, —OCH₂CO₂R¹⁰, —CH₂CO₂R¹⁰, —CH₂SR¹⁰, —CH₂NR¹⁰R¹⁰, —CH₂CONR¹⁰R¹⁰, —SR¹⁰—OSR¹⁰, —N(R¹⁰)(R¹⁰), or —NR¹⁰SO₂R¹⁰,
   where each R¹⁰ is independently selected from hydrogen, (C₁-C₁₀) alkyl, (C₃-C₁₀) cycloalkyl, —SCX₃ in which X is a halogen, —CN, —NO₂ or -Z-A-Z′- in which Z and A are as defined above and Z′ represents carbon, oxygen, sulfur, or nitrogen;
      wherein R⁴ is H, OH, halide, unsubstituted or substituted, straight or branched (C₁-C₅) alkyl group, (C₂-C₅) alkenyl, or a (C₂-C₅) alkynyl, (C₁-C₅) alkoxy, (C₂-C₅) alkenyloxy, or (C₂-C₅) alkynyloxy, —N₃, —C(O)R¹¹, —C(O)NR¹¹R¹², C(O)OR¹¹, —OC(O)R¹¹, —OC(O)OR¹¹, —NH(OH), —N(R¹¹)(R¹²), —N(H)COR¹¹, —N(OH)COR¹¹, —CH₂OR¹¹, —OCH₂CO₂R¹¹, —CH₂SR¹¹, —CH₂N(R¹¹)(R¹²), —SR¹¹, —OSR¹¹, —N(R¹¹) SO₂R¹², —OR¹³ or triethylsiloxy,
where R¹³ is H, —C(O)—O—R¹⁴, or —C(O)(R¹⁴), where R¹⁴ is alkyl, aryl, or amino, and where R¹¹ and R¹² are each, independently, hydrogen, substituted or unsubstituted (C₁-C₅) alkyl, (C₂-C₅) alkenyl, (C₂-C₅) alkynyl, or cycloalkyl or aryl group having 3 to 10 carbon atoms; and
   wherein R¹⁵ is H or halide, and R¹⁶ is —C(CH₃)—C(O)—OCH₃,
   to NaBH₄ in a suitable solvent so as to produce a second compound having the structure:

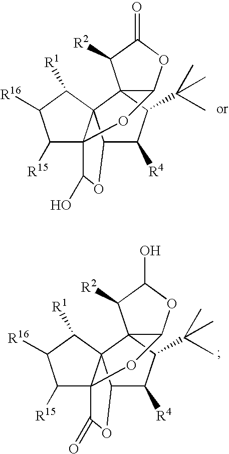

(b) reacting the lactol product of step (a) with an agent suitable to produce a compound having the structure:

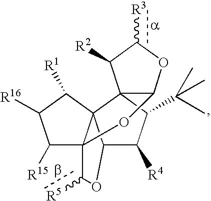

wherein R³ is O and R⁵ is selected from H, OH, —C(CH₃)—C(O)—O(CH₃), (C₁-C₁₀) alkyl, (C₂-C₁₀) alkenyl, (C₂-C₁₀) alkynyl, substituted or unsubstituted aryl, substituted or unsubstituted heteroaryl, heterocyclic, amino, amido, alkoxy, alkenyloxy, alkynyloxy, —O-aryl, —O-(alkyl)(C₁-C₁₀), (—NH-alkyl, —N(alkyl)₂, —NH₂, -alkyl-C(O)(OH), -alkyl-OH, -alkyl-(NH₂), halide, CX₃ where X is a halide, indole radical, -A-Ar, -A-Z-Ar, —SO₂—Ar, -A-NR¹⁰, —O-A-Ar, or R¹⁰, where A, Z, Ar and R¹⁰ are defined as above, or
wherein R⁵ is O and R³ is selected from OH, —C(CH₃)—C(O)—O(CH₃), (C₁-C₁₀) alkyl, (C₂-C₁₀) alkenyl, (C₂-C₁₀) alkynyl, substituted or unsubstituted aryl, substituted or unsubstituted heteroaryl, heterocyclic, amino, amido, alkoxy, alkenyloxy, alkynyloxy, —O-aryl, —O—(C₂-C₁₀ alkyl) (C₁-C₁₀), (—NH-alkyl, —N(alkyl)₂, —NH₂, -alkyl-C(O) (OH), -alkyl-OH, -alkyl-(NH₂), halide, CX₃ where X is a halide, indole radical, -A-Ar, -A-Z-Ar, —SO₂—Ar, -A-NR¹⁰, —O-A-Ar, or —R¹⁰, where A, Z, Ar and R¹⁰ are defined as above; and
(c) joining R¹⁶ and R¹⁵ to form a lactone.

This invention further provides the instant process, wherein the suitable solvent in step (a) is MeOH.

This invention further provides the instant process, wherein step (a) is performed at room temperature.

This invention further provides the instant process, further comprising the step of exposing the compound produced in step (a) to p-phenylbenzoic acid, EDC and DMAP so as to resolve the enantiomers before step (b).

This invention further provides the instant process, wherein the suitable agent is a carboxcylic acid, an alkylating reagent or an acid halide. In one embodiment the alkylating reagent is an alkyliodide. In one embodiment the alkylating reagent is methyliodide.

This invention also provides a process for preparing a compound having the structure:

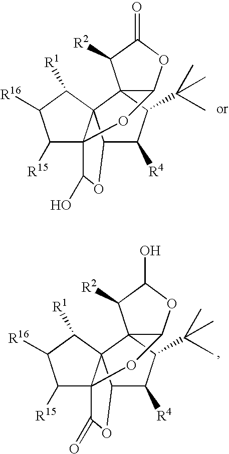

comprising reacting

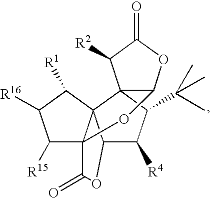

wherein R¹ is H or —OR⁸,
wherein R² is H, OH, —O(CH₃), —OC(O)CH₃, (C₁-C₁₀) alkyl, (C₂-C₁₀) alkenyl, (C₂-C₁₀) alkynyl, -A-Ar, -A-Z-Ar, —SO₂—Ar, -A-NR¹⁰, —O-A-Ar, or —R¹⁰,
where A is (C₁-C₈) alkyl, (C₂-C₈) alkenyl, (C₂-C₈) alkynyl, which is unsubstituted or substituted by a straight or branched alkyl chain group having 1 to 5 carbon atoms; Z is carbon, oxygen, sulfur or nitrogen; Ar is a phenyl group, a pyridyl group, a naphthyl group, a pyrimidyl group, or a quinolyl group, each of which is unsubstituted or substituted by one to five substituents selected from the group consisting of hydrogen, halogen, a hydroxy group, a carboxylic acid group, substituted or unsubstituted (C₁-C₁₀) alkyl, (C₂-C₁₀) alkenyl, (C₂-C₁₀) alkynyl, (C₁-C₁₀) haloalkyl, (C₁-C₁₀) alkoxy, (C₂-C₁₀) alkenyloxy, (C₂-C₁₀) alkynyloxy, (C₁-C₁₀) haloalkoxy, a phenyl group, a phenoxy group, an aralkyl group, an aralkyloxy group, a substituted phenyl group, a substituted phenoxy group, a substituted aralkyl group, a substituted aralkyloxy group, —C(O)R¹⁰, —C(O)NR¹⁰R¹⁰, —C(O)OR¹⁰, —N(H)COR¹⁰, —NH(OH), —N(OH)COR¹⁰, —CH₂OR¹⁰, —OCH₂CO₂R¹⁰, —CH₂CO₂R¹⁰, —CH₂SR¹⁰, CH₂NR¹⁰R¹⁰, —CH₂CONR¹⁰R¹⁰, —SR¹⁰, —OSR¹⁰, —N(R¹⁰)(R¹⁰) or —NR¹⁰SO₂R¹⁰,
   where each R¹⁰ is independently selected from hydrogen, (C₁-C₁₀) alkyl, (C₃-C₁₀) cycloalkyl, —SCX₃ in which X is a halogen, —CN, —NO₂ or -Z-A-Z′- in which Z and A are as defined above and Z′ represents carbon, oxygen, sulfur, or nitrogen;
      wherein R⁴ is H, OH, halide, unsubstituted or substituted, straight or branched (C₁-C₅) alkyl group, (C₂-C₅) alkenyl, or a (C₂-C₅) alkynyl, (C₁-C₅) alkoxy, (C₂-C₅) alkenyloxy, or (C₂-C₅) alkynyloxy, —N₃, —C(O)R¹¹, —C(O)NR¹¹R¹², —C(O)OR¹¹, —OC(O)R¹¹, —OC(O)OR¹¹, —NH(OH), —N(R¹¹)(R¹²), —N(H)COR¹¹, —N(OH)COR¹¹, —CH₂OR¹¹, —OCH₂CO₂R¹¹, —CH₂SR¹¹, —CH₂N(R¹¹)(R¹²), —SR¹¹, —OSR¹¹, —N(R¹¹)SO₂R¹², —OR¹³, or triethylsiloxy,
where R¹³ is H, —C(O)—O—R¹⁴, or —C(O)(R¹⁴), where R¹⁴ is alkyl, aryl, or amino, and where R¹¹ and R¹² are each, independently, hydrogen, substituted or unsubstituted (C₁-C₅) alkyl, (C₂-C₅) alkenyl, (C₂-C₅) alkynyl, or cycloalkyl or aryl group having 3 to 10 carbon atoms; and
   wherein R¹⁵ is H or halide, and R¹⁶ is —C(CH₃)—C(O)—OCH₃,
   with NaBH₄ in a suitable solvent.

This invention further provides the instant process, wherein the compound produced is:

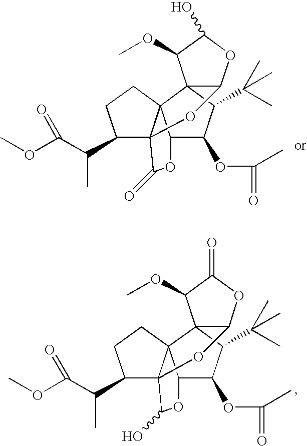

and the process comprises reacting:

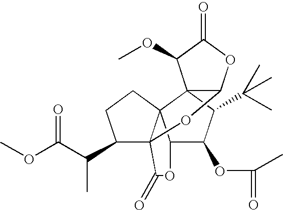

with NaBH₄ in a suitable solvent.

This invention further provides the instant process, wherein the suitable solvent in step (a) is MeOH.

This invention further provides the instant process, wherein step (a) is performed at room temperature.

This invention also provides a process for preparing a compound having the structure:

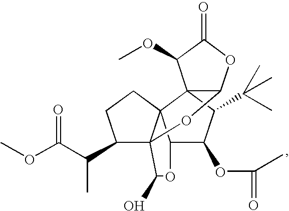

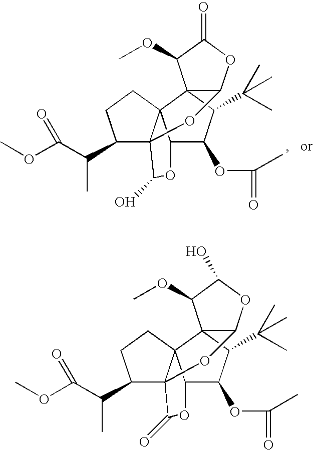

comprising:
a) exposing a compound having the structure:

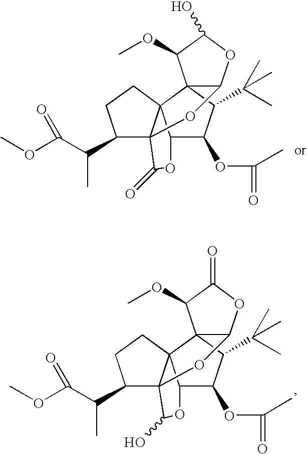

to p-phenylbenzoic acid, DEC, DMAP, at a suitable temperature so as to produce a compound having the structure:

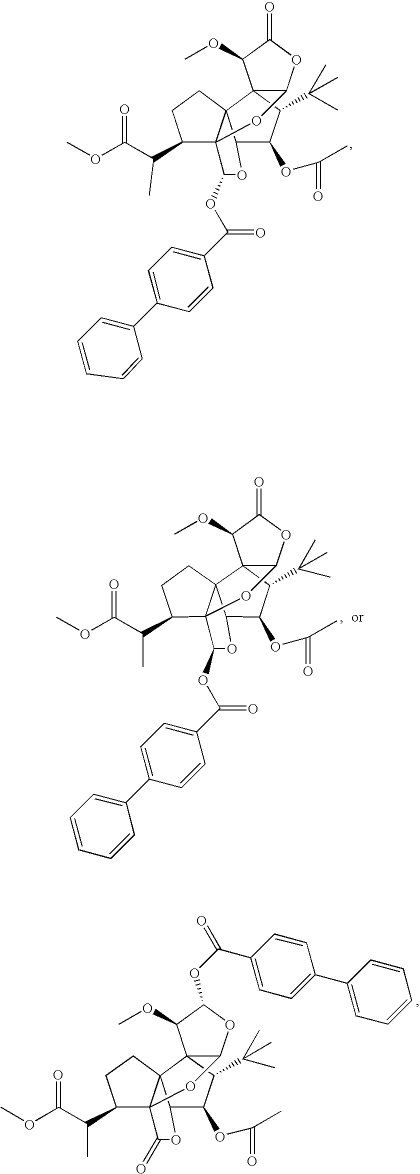

b) separating the compounds produced in step a); and
c) exposing the products of step b) to a suitable hydrolyzing agent so as to produce the compound.

This invention further provides the instant process, wherein the hydrolyzing agent is K₂CO₃ in a suitable solvent.

This invention further provides the instant process, wherein the products of step b) are separated using silica gel thin layer chromatography.

This invention further provides the instant process, wherein step (a) is performed at room temperature

This invention also provides a method of making a composition comprising admixing an effective amount of a compound of any one of the instant compounds and a pharmaceutically acceptable carrier.

This invention also provides a composition comprising any one of the instant compounds and a carrier.

The ginkgolide lactol derivatives and f-seco ginkgolide lactol derivatives disclosed here are expected to be useful antagonists against the platelet activating factor receptor (PAFR) and of the inhibitory glycine and GABA_{A} receptors.

As used in the structural diagrams herein, a wavy line bond denotes a bond that has variable 3-D geometry, i.e either comes out of, or goes into, the plane of the paper.

As used herein, “room temperature” means between 18° C. and 27° C., and more preferably 20-25° C.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. For example, an amount effective to inhibit or reverse depressive disorder or anxiety disorder symptoms, or for example to inhibit, attenuate or reverse disorder symptoms. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, a “salt” is salt of the instant compounds which has been modified by making acid or base salts of the compounds. In the case of compounds used for treatments, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium.

As used herein, a “pharmaceutically acceptable carrier” is a pharmaceutically acceptable solvent, suspending agent or vehicle, for delivering the instant compounds to an animal or human. The carrier may be liquid or solid and is selected with the planned manner of administration in mind. Liposomes are also a pharmaceutical carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds may comprise a single compound or mixtures thereof with other compounds. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or other methods, all using dosage forms well known to those of ordinary skill in the pharmaceutical arts.

The compounds can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The active ingredient can be administered orally in solid dosage forms, such as capsules, tablets, and powders, or in liquid dosage forms, such as elixirs, syrups, and suspensions. It can also be administered parentally, in sterile liquid dosage forms.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The instant compounds may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The present invention also includes pharmaceutical kits, which comprise one or more containers containing a pharmaceutical composition comprising an effective amount of one or more of the compounds. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2, . . . , n−1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, propyl, butyl, pentyl, hexyl, and so on. “Alkoxy” represents an alkyl group, which may have an indicated number of carbon atoms, attached through an oxygen bridge.

The term “alkyl” as used in the terms “-alkyl-OH”, “—NH-alkyl”, “-alkyl-(NH₂)”, “-alkyl-C(O)(OH)”, and “—O-alkyl” are C₁-C₆ alkyl as defined above.

The term “alkyl” as used in the term “—N(alkyl)₂” means alkyl as defined above. However, the two alkyl groups of “—N(alkyl)₂” need not necessarily be the same type of alkyl group. For example one alkyl may be chosen from the group methyl, ethyl, propyl, butyl, pentyl, or hexyl in a linear or branched arrangement unless otherwise specified and the other alkyl may be independently chosen from the group methyl, ethyl, propyl, butyl, pentyl, or hexyl.

The term “cycloalkyl” shall mean cyclic rings of alkanes of three to ten total carbon atoms, or any number within this range (i.e., cyclopropyl, cyclobutyl, cyclopentyl, cyclohexyl, cycloheptyl or cyclooctyl).

If no number of carbon atoms is specified, the term “alkenyl” refers to a non-aromatic hydrocarbon radical, straight or branched unless otherwise specified, containing at least 1 carbon to carbon double bond, and up to the maximum possible number of non-aromatic carbon-carbon double bonds may be present. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. With regard to “alkenyl”, R¹ through R⁶ as used here are C₂-C₆

The term “cycloalkenyl” shall mean cyclic rings of 3 to 10 carbon atoms and at least 1 carbon to carbon double bond (i.e., cyclopropenpyl, cyclobutenyl, cyclopenentyl, cyclohexenyl, cycloheptenyl or cycloocentyl).

The term “alkynyl” refers to a hydrocarbon radical straight or branched, containing at least 1 carbon to carbon triple bond, and up to the maximum possible number of non-aromatic carbon-carbon triple bonds may be present. Thus, “C₂-C₆ alkynyl” means an alkynyl radical radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. With regard to “alkynyl”, R² through R⁶ as used here are C₂-C₆.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. The substituted aryls included in this invention include substitution at any suitable position with amines, substituted amines, alkylamines, hydroxys and alkylhydroxys, wherein the “alkyl” portion of the alkylamines and alkylhydroxys is a C₂-C₆ alkyl as defined hereinabove. The substituted amines may be substituted with alkyl, alkenyl, alkynl, or aryl groups as hereinabove defined.

The term “heteroaryl”, as used herein, represents a stable monocyclic or bicyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As appreciated by those of skill in the art, “halo”, “halide”, or “halogen” as used herein means chloro, fluoro, bromo or iodo.

The term “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

The alkyl, alkenyl, alkynyl, cycloalkyl, aryl, heteroaryl and heterocyclyl substituents may be unsubstituted or unsubstituted, unless specifically defined otherwise. For example, a (C₁-C₆) alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, alkynyl, cycloalkyl, cycloalkenyl, heterocyclyl and heteroaryl groups can be further substituted by replacing one or more hydrogen atoms be alternative non-hydrogen groups. These include, but are not limited to, halo, hydroxy, mercapto, amino, carboxy, cyano and carbamoyl.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing compounds of the present invention, one of ordinary skill in the art will recognize that the various substituents, i.e. R¹ through R¹⁶ are to be chosen in conformity with well-known principles of chemical structure connectivity.

This invention will be better understood by reference to the Experimental Details which follow, but those skilled in the art will readily appreciate that the specific experiments detailed are only illustrative of the invention as described more fully in the claims which follow thereafter.

### Experimental Details

### F-Seco-Ginkgolides

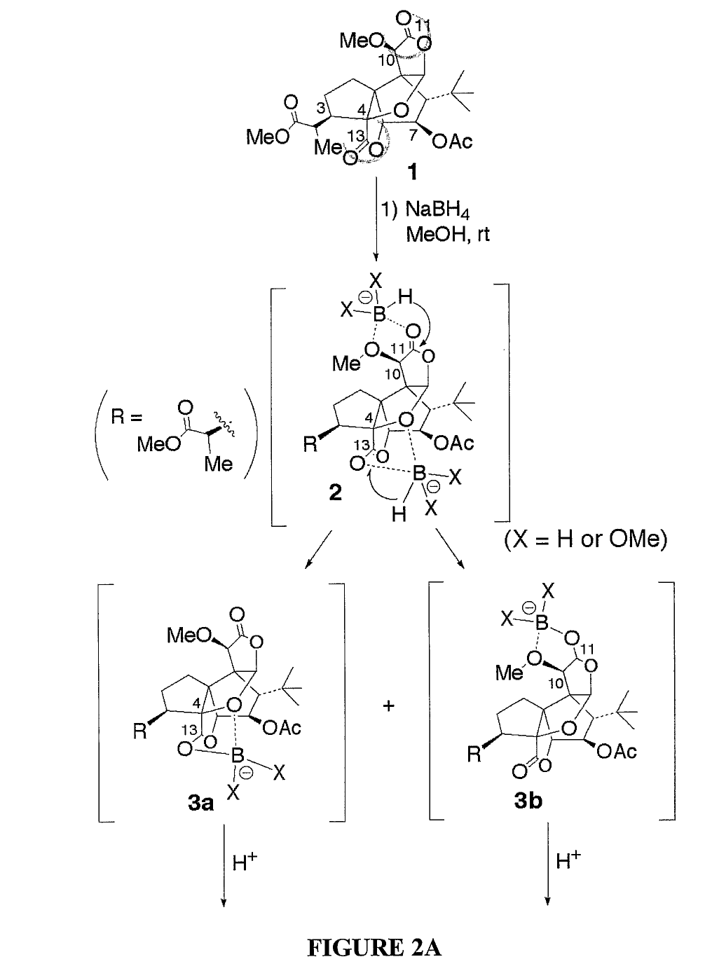

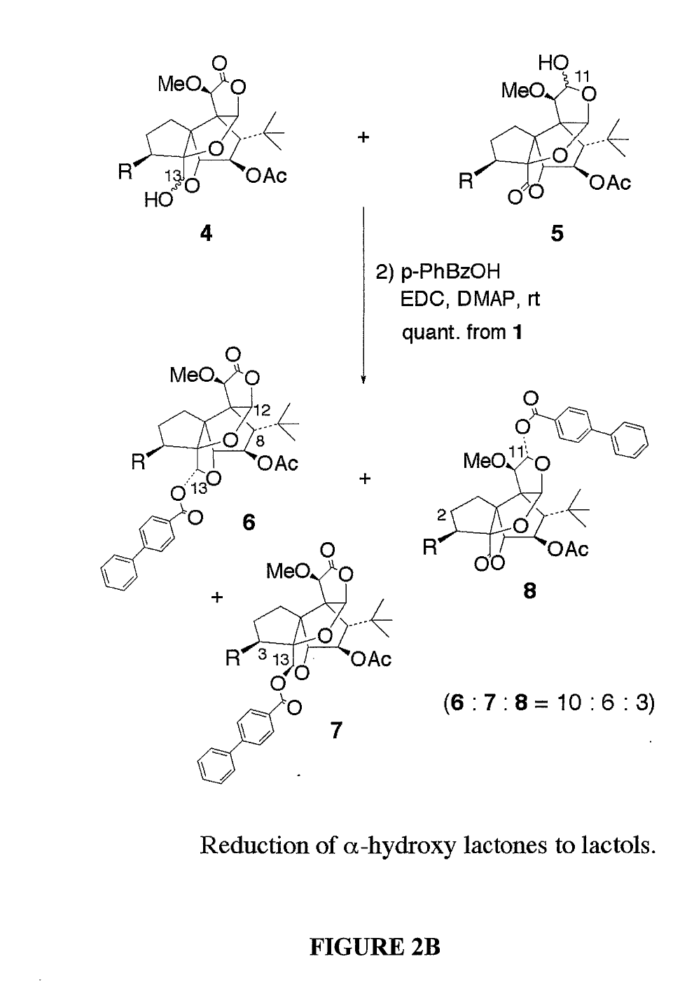

In addition to the two C-10 and C-7 hydroxyls, the presence of the unusual C-3 ester group renders 1 a unique ginkgolide template, see FIG. 2. Unexpectedly, it was found that the α-hydroxyl lactone moieties in 1 are readily reduced by sodium borohydride (NaBH₄) to produce the corresponding lactols (FIG. 2). The unique reactivity of NaBH₄ towards the α-hydroxyl lactone moieties of ginkgolide and its derivatives, permitted synthesis of a number of derivatives.

### NaBH₄ Treatment

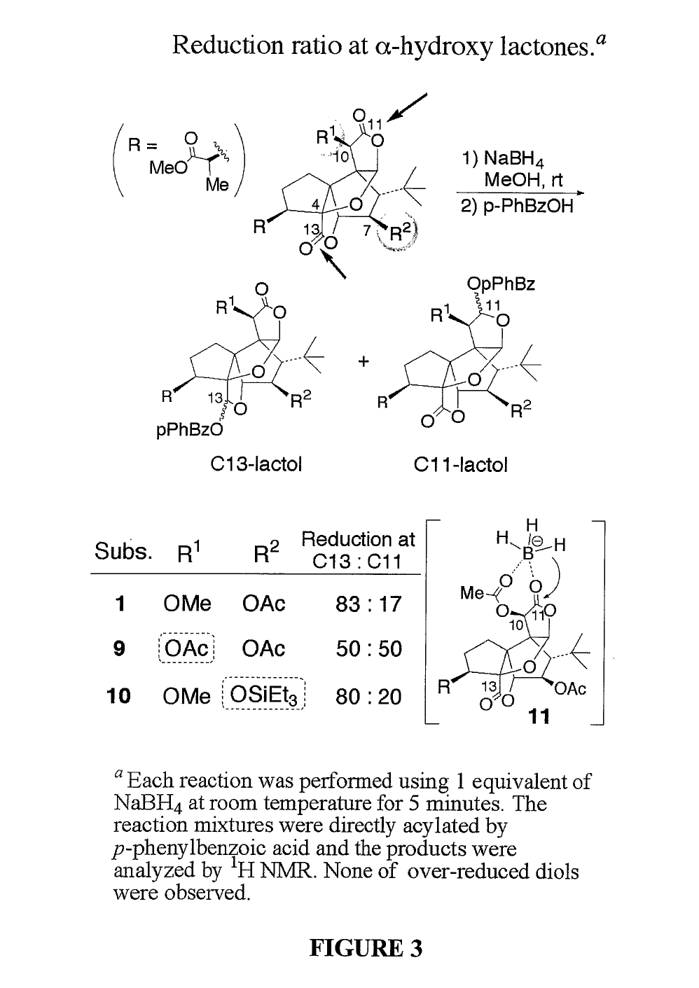

NaBH₄ treatment (1 equivalent) of 1 quantitatively provided the C-13 and C-11 lactol derivatives 4 and 5 (FIG. 2). The reaction was completed within 5 minutes at room temperature, and interestingly, none of the over-reduced dialcohols were obtained even upon exposure to excess NaBH₄ and/or prolonged reaction time. It is to be noted that the ester group in 1 was not reduced under these conditions. Since lactol derivatives 4 and 5 exist as 1:1 equilibrium mixtures of lactol hydroxy groups at both C-11 and C-13, the isolation and separation became possible only after acylation. Thus, treatment of 4 and 5 with p-phenylbenzoic acid in the presence of EDC and DMAP gave 6-8 in a ratio of 10:6:3 (27) each isomer being readily separable by silica gel TLC (28); the chemoselectivity ratio of reduction at C-13 and C-11 were thus 83:17, respectively (see FIG. 3).

The stereochemistry of the 11- and two 13-p-phenylbenzoate, 6-8, were assigned from the following NOEs: 13-H/8-H and 13-H/12-H for 13α-benzoate 6, 13-H/3-H for 13β-benzoate derivative 7, and 2-H/11-H for 8. The configuration of the main isomer 6 was also confirmed by new cross metathesis/CD and/or FDCD exciton chirality protocol (29).

Our studies revealed that the ginkgolide α-hydroxyl lactones are converted smoothly, selectively, and quantitatively into lactols, by reacting with 1 equivalent NaBH₄ at room temperature for a few minutes (30). In contrast, it is well known that the reduction of lactones or esters by NaBH₄ requires a large excess of the reagent, i.e., exceeding 20 equivalents, and/or relatively high reaction temperatures (31-34). Furthermore, when such reduction of lactones proceeds, in most cases the products are the diols resulting from over-reduction of the intermediary lactols, as is the case of polyhydroxylated sugar lactones.

It has been reported that the electron withdrawing α-oxygen or coordinating functionalities linked to the carbonyl groups, e.g. (α-amino acids, accelerate the NaBH₄ reduction (32,35,36). A unique reactivity of ginkgolide lactones is therefore most likely caused by the presence of suitably arranged C-4 and C-10 α-oxygens which are rigidly fixed in the ginkgolide cage-shaped skeleton (FIG. 2). Namely, NaBH₄ presumably coordinates tightly with the lactone carbonyls and α-oxygens to yield a complex such as 2 that could accelerate the nucleophilic attack of the hydride towards the lactone carbonyl, which in turn is activated by the hydroxyl inductive effect. The preferred reduction of the 13-lactone (C-13: C-11=83:17) is most likely due to the stronger coordination of NaBH₄ to this carbonyl. In addition, the obtained lactol hydroxyl and α-hydroxyl could form a strong borate complex such as 3a and 3b which might stabilize the reaction intermediates and prevent further reduction, a phenomenon similar to the well-known partial reduction of lactones by diisobutyl aluminum hydride (DIBAL) at low temperature, i.e., −78° C. Piancatelli and co-worker have also found that glycidic lactones (α-epoxy lactones) are readily reduced to glycidic lactols by NaBH₄, although the latter are gradually reduced further to diols upon a prolonged reaction period (37). Note that the DIBAL reduction of 1 leads to a mixture of products; the mild NaBH₄ reduction is thus an efficient alternative to obtain the α-hydroxy lactol derivatives.

### Substituent Effects

We further examined the substituent effects on the NaBH₄ reduction at C-7 and C-10 of 1 (FIG. 3). Interestingly, when the C-10 methoxy substituent of 1 (R1 substituent) was replaced by the acetoxy group in 9, the reduction ratio at C-11 carbonyl increased (C-13:C-11=50:50), possibly due to better coordination of NaBH₄ with the α-acetoxy lactone moiety, which increases the reactivity at C-11 carbonyl (see structure 11). In contrast, NaBH₄ treatment of 10, in which the 7-acetoxy group in 1 (R2 substituent) was replaced by the bulkier triethylsiloxy group, provided a C-11 to C-13 lactol ratio similar to that obtained for 1 (C-13:C-11=80:20), indicating that the remote C-7 substituents exert no steric and/or electronic influence.

### Natural Ginkgolides

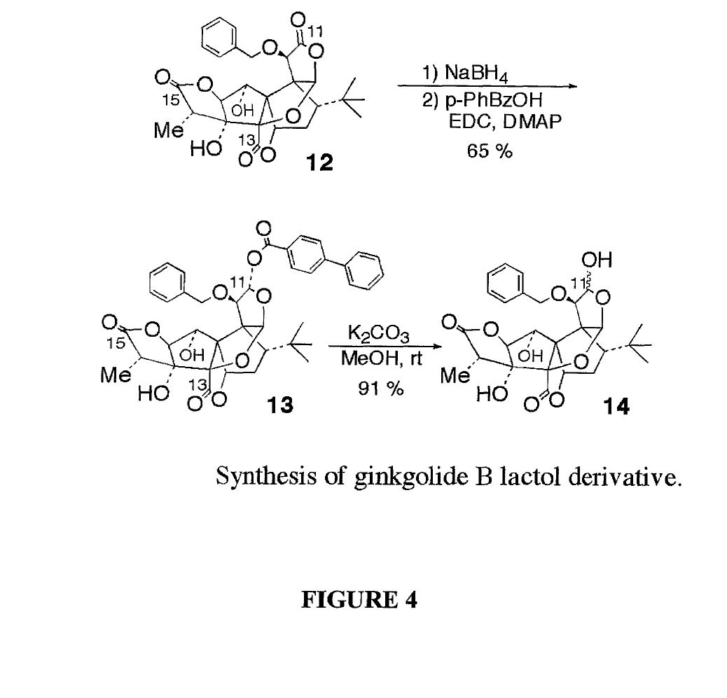

The method was further applied to the natural ginkgolides (FIG. 4). α-Benzyl ginkgolide B (12), the most potent ginkgolide antagonist against PAF receptor (1), was readily reduced by NaBH₄ to give C-11 lactol derivative 13 as the major product, which was separated from the minor C-13 lactol by acylation with p-phenylbenzoic acid. It is noted that the reduction did not proceed at the C-15 lactone that lacks a α-hydroxyl function. Phenylbenzoate 13 was hydrolysed to lactol 14 with K₂CO₃ in 91% yield. Similarly, the hydrolysis of p-phenylbenzoate derivatives obtained in FIG. 2 and FIG. 3 readily yielded an equilibrium mixture of the corresponding lactols. The efficient NaBH₄ reduction of 1 and 9-12 thus provided a variety of ginkgolide lactols and their diastereomeric acylates leading to a total of 25 acylated or alkylated derivatives at 3- and 7-hydroxyl.

Derivatization of C₁₁ and/or C₁₃

The C₁₁/C₁₃ lactol derivatives of the f-seco-ginkgolides and natural ginkgolides disclosed here may readily be derivatized at the C₁₁/Cl₃ position using known techniques. Examples of such techniques are given in U.S. Pat. No. 6,693,091 and U.S. Patent Application Publication No. US 2003-0225052 A1, each of which documents are hereby incorporated by reference.

### Materials and General Methods

### Representative Procedure of Ginkgolide Lactol Benzoates:

To a solution of ginkgolide derivatives (ca. 0.05 mmol) in MeOH (1 mL) was added NaBH₄ (1 equivalent) at room temperature, and the mixture was stirred for 5 min. The reaction mixture was directly subjected to rapid chromatography on silica gel (50% ethyl acetate in hexane) to afford the corresponding lactol derivatives. To a solution of the lactol mixture obtained above in dichloromethane (1 mL) was added p-phenylbenzoic acid (2 equivalents), EDC (2.2 equivalents), and DMAP (2.2 equivalents) at room temperature, and the mixture was stirred for 12 h. The reaction mixture was concentrated in vacuo to give the crude products which were purified by preparative thin layer chromatography on silica gel to afford the lactol p-phenylbenzoate derivatives.

Data for 6; ¹H NMR (300 MHz, CDCl₃) δ 1.11 (s, 9H), 1.15 (d, 3H, J=7.2 Hz), 1.77-1.98 (m, 2H), 2.07 (s, 3H), 2.21-2.28 (m, 1H), 2.57 (d, 1H, J=12.3 Hz), 2.69-2.80 (m, 1H), 2.93-3.05 (m, 2H), 3.59 (s, 3H), 3.77 (s, 3H), 4.47 (d, 1H, J=3.3 Hz), 4.58 (s, 1H), 5.14 (dd, 1H, J=12.3, 3.3 Hz), 5.87 (s, 1H), 6.44 (s, 1H), 7.36-7.48 (m, 3H), 7.61 (d, 2H, J=7.2 Hz), 7.68 (d, 2H, J=8.4 Hz), 8.09 (d, 2H, J=8.4 Hz); HRFABMS calculated for C₃₇H₄₃O₁₁ [M+H]⁺ 663.2805. found 663.2813.

Data for 7; ¹H NMR (300 MHz, CDCl₃) δ 1.05 (s, 9H), 1.24 (d, 3H, J=7.2 Hz), 1.74-1.98 (m, 3H), 2.00 (s, 3H), 2.37-2.46 (m, 1H), 2.85-2.96 (m, 2H), 3.06 (d, 1H, J=12.3 Hz), 3.63 (s, 3H), 3.73 (s, 3H), 4.50 (s, 1H), 4.54 (d, 1H, J=6.0 Hz), 5.01 (dd, 1H, J=12.3, 6.0 Hz), 6.04 (s, 1H), 6.36 (s, 1H), 7.40-7.52 (m, 3H), 7.65 (d, 2H, J=7.2 Hz), 7.70 (d, 2H, J=8.4 Hz), 8.18 (d, 2H, J=8.4 Hz); HRFABMS calculated for C₃₇H₄₃O₁₁ [M+H]⁺ 663.2805. found 663.2810.

Data for 8; ¹H NMR (300 MHz, CDCl₃) δ 1.16 (s, 9H), 1.23 (d, 3H, J=6.9 Hz), 1.91-1.95 (m, 1H), 2.05-2.13 (m, 3H), 2.13 (s, 3H), 2.51-2.66 (m, 2H), 2.90-3.01 (m, 1H), 3.40 (s, 3H), 3.72 (s, 3H), 4.66 (d, 1H, J=7.2 Hz), 4.67 (s, 1H), 5.12 (dd, 1H, J=12.9, 4.5 Hz), 5.95 (s, 1H), 6.58 (d, 1H, J=3.3 Hz), 7.40-7.52 (m, 3H), 7.63 (d, 2H, J=7.2 Hz), 7.71 (d, 2H, J=8.4 Hz), 8.10 (d, 2H, J=8.4 Hz); HRFABMS calculated for C₃₇H₄₃O₁₁ [M+H]⁺ 663.2805. found 663.2816.

Data for 13; ¹H NMR (300 MHz, CDCl₃) δ 1.20 (s, 9H), 1.28 (d, 3H, J=6.9 Hz), 1.93-1.96 (m, 2H), 2.24-2.35 (m, 1H), 2.76 (s, 1H, C3-OH), 3.00 (d, 1H, J=3.0 Hz, C10-OH), 3.54 (q, 1H, J=6.9 Hz), 4.43 (dd, 1H, J=8.1, 3.3 Hz), 4.52 (d, 1H, J=9.6 Hz), 4.58 (d, 1H, J=7.8 Hz), 4.69 (d, 1H, J=9.9 Hz), 5.04 (d, 1H, J=2.4 Hz), 5.36 (d, 1H, J=3.0 Hz), 6.00 (s, 1H), 6.73 (d, 1H, J=2.4 Hz), 7.30-7.34 (m, 2H), 7.37-7.53 (m, 6H), 7.63-7.66 (m, 2H), 7.73 (d, 2H, J=8.4 Hz), 8.10 (d, 2H, J=8.4 Hz); HRFABMS calculated for C₄₀H₄₁O₁₁ [M+H]⁺ 697.2649. found 697.2659.

### REFERENCES

1. Stromgaard, K.; Nakanishi, K. Angew. Chem. Int. Ed. 2004, 43, 1640-1658.
2. Braquet, P.; Drieu, K.; Etienne, A. Actual. Chim. Ther. 1986, 13, 237-254.
3. Braquet, P.; Spinnewyn, B.; Braquet, M.; Bourgain, R. H.; Taylor, J. E.; Etienne, A.; Drieu, K. Blood Vessels 1985, 16, 558-572.
4. Kondratskaya, E. L.; Lishko, P. V.; Chatterjee, S. S.; Krishtal, O. A. Neurochem. Int. 2002, 40, 647-653.
5. Kondratskaya, E. L.; Krishtal, I. A. Neurophysiology 2002, 34, 155-157.
6. Ivic, L.; Sands, T. T. J.; Fishkin, N.; Nakanishi, K.; Kriegstein, A. R.; Stromgaard, K. J. Biol. Chem. 2003, 278, 49279-49285.
7. Rapin, J. R.; Zaibi, M.; Drieu, K. Drug Dev. Res. 1998, 45, 23-29.
8. Braquet, P. Drugs Future 1987, 12, 643-699.
9. Braquet, P.; Esanu, A.; Buisine, E.; Hosford, D.; Broquet, C.; Koltai, M. Med. Res. Rev. 1991, 11, 295-355.
10. Hu, L.; Chen, Z.; Xie, Y. J. Asian Nat. Prod. Res. 2001, 4, 219-227.
11. Hu, L.; Chen, Z.; Xie, Y.; Jiang, Y.; Zhen, H. J. Asian Nat. Prod. Res. 2000, 3, 103-110.
12. Hu, L.; Chen, Z.; Xie, Y.; Jiang, H.; Zhen, H. Bioorg. Med. Chem. 2000, 8, 1515-1521.
13. Corey, E. J.; Gavai, A. V. Tetrahedron Lett. 1989, 30, 6959-6962.
14. Corey, E. J.; Rao, K. S. Tetrahedron Lett. 1991, 32, 4623-4626.
15. Park, H. K.; Lee, S. K.; Park, P. U.; Kwak, W. J. In Sunkyong Industries Co., Ltd., S. Korea 1993, p WO 9306107.
16. Park, P.-U.; Pyo, S.; Lee, S.-K.; Sung, J. H.; Kwak, W. J.; Park, H.-K.; Cho, Y.-B.; Ryu, G.-H.; Kim, T. S. In Sunkyong Industries Co., Ltd., S. Korea 1995, p WO 9518131.
17. Stromgaard, K.; Saito, D. R.; Shindou, H.; Ishii, S.; Shimizu, T.; Nakanishi, K. J. Med. Chem. 2002, 45, 4038-4046.
18. Jaracz, S.; Stromgaard, K.; Nakanishi, K. J. Org. Chem. 2002, 67, 4623-4626.
19. Jaracz, S.; Nakanishi, K.; Jensen, A. A.; Stromgaard, K. Chem. Eur. J. 2004, 10, 1507-1518.
20. Maruyama, M.; Terahara, A.; Itagaki, Y.; Nakanishi, K. Tetrahedron Lett. 1967, 4, 299-303.
21. Maruyama, M.; Terahara, A.; Itagaki, Y.; Nakanishi, K. Tetrahedron Lett. 1967, 4, 303-308.
22. Maruyama, M.; Terahara, A.; Nakadaira, Y.; Woods, M. C.; Nakanishi, K. Tetrahedron Lett. 1967, 4, 309-313.
23. Maruyama, M.; Terahara, A.; Nakadaira, Y.; Woods, M. C.; Takagi, Y.; Nakanishi, K. Tetrahedron Lett. 1967, 4, 315-319.
24. Woods, M. C.; Miura, I.; Nakadaira, Y.; Terahara, A.; Maruyama, M.; Nakanishi, K. Tetrahedron Lett. 1967, 4, 321-326.
25. Nakanishi, K. Pure Appl. Chem. 1967, 14, 89-113.
26. Maruyama, M.; Terahara, A. The Science Reports of the Tohoku University 1967, L, 92-99.
27. Under this condition, bis-lactol derivatives were obtained less than 10% of the products.
28. Corey, E. J.; Albonico, S. M.; Koelliker, U.; Schaaf, T. K.; Varma, R. K. J. Am. Chem. Soc. 1971, 93, 1491-1493.
29. Tanaka, K.; Pescitelli, G.; Nakanishi, K.; Berova, N. Monatsh. Chem., in press.
30. Our control experiment shows that the lactone without a-hydroxyls, i.e., Corey lactone is not reduced under the condition employed in Scheme 1.
31. Soai, K.; Oyamada, H.; Ookawa, A. Synth. Commun. 1982, 12, 463-467.
32. Barrett, A. G. M. Reduction of carboxylic acid derivatives to alcohols, ethers and amines; Pergamon Press: London, 1991; Vol. 5, Chapter 6.2.
33. Wolfrom, M. L.; Wood, H. B. J. Am. Chem. Soc. 1951, 73, 2933-2934.
34. Wolfrom, M. L.; Anno, K. J. Am. Chem. Soc. 1952, 74, 5583-5584.
35. Barnett, J. E. G.; Kent, P. W. J. Am. Chem. Soc. 1963, 85, 2743-2747.
36. Mauger, J.; Robert, A. J. Chem. Soc., Chem. Commun. 1986, 395-396.
37. Corsano, S.; Piancatelli, G. J. Chem. Soc., Chem. Commun. 1971, 1106.

1. A compound having the structure: 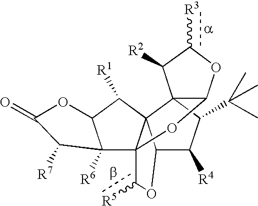
wherein R¹ is H or —OR⁸,
where R⁸ is H, or —C(O)R⁹, where R⁹ is alkyl, aryl, or amino;
wherein R² is H, OH, —O(CH₃), —OC(O)CH₃, (C₁-C₁₀) alkyl, (C₂-C₁₀) alkenyl, (C₂-C₁₀) alkynyl, -A-Ar, -A-Z-Ar, —SO₂—Ar, -A-NR¹⁰, —O-A-Ar, or —R¹⁰,
where A is (C₁-C₈) alkyl, (C₂-C₈) alkenyl, (C₂-C₈) alkynyl, which is unsubstituted or substituted by a straight or branched alkyl chain group having 1 to 5 carbon atoms; Z is carbon, oxygen, sulfur or nitrogen; Ar is a phenyl group, a pyridyl group, a naphthyl group, a pyrimidyl group, or a quinolyl group, each of which is unsubstituted or substituted by one to five substituents selected from the group consisting of hydrogen, halogen, a hydroxy group, a carboxylic acid group, substituted or unsubstituted (C₁-C₁₀) alkyl, (C₂-C₁₀) alkenyl, (C₂-C₁₀) alkynyl, (C₁-C₁₀) haloalkyl, (C₁-C₁₀) alkoxy, (C₂-C₁₀) alkenyloxy, (C₂-C₁₀) alkynyloxy, (C₁-C₁₀) haloalkoxy, a phenyl group, a phenoxy group, an aralkyl group, an aralkyloxy group, a substituted phenyl group, a substituted phenoxy group, a substituted aralkyl group, a substituted aralkyloxy group, —C(O)R¹⁰, C(O)NR¹⁰R¹⁰, —C(O)OR¹⁰, —N(H)COR¹⁰—NH(OH), —N(OH)COR¹⁰, —CH₂OR¹⁰, —OCH₂CO₂R¹⁰, —CH₂CO₂R¹⁰, —CH₂SR¹⁰, —CH₂NR¹⁰R¹⁰, —CH₂CONR¹⁰R¹⁰, —SR¹⁰—OSR¹⁰, —N(R¹⁰)(R¹⁰) or —NR¹⁰SO₂R¹⁰,
where each R¹⁰ is independently selected from hydrogen, (C₁-C₁₀) alkyl, (C₃-C₁₀) cycloalkyl, —SCX₃ in which X is a halogen, —CN, —NO₂ or -Z-A-Z′- in which Z and A are as defined above and Z′ represents carbon, oxygen, sulfur, or nitrogen;
wherein R⁴ is H, OH, halide, unsubstituted or substituted, straight or branched (C₁-C₅) alkyl group, (C₂-C₅) alkenyl, or a (C₂-C₅) alkynyl, (C₁-C₅) alkoxy, (C₂-C₅) alkenyloxy, or (C₂-C₅) alkynyloxy, —N₃, —C(O)R¹¹, —C(O)NR¹¹R¹², —C(O)OR¹¹, —OC(O)R¹¹, —OC(O)OR¹¹, —NH(OH), —N(R¹¹)(R¹²), —N(H)COR¹¹, —N(OH)COR¹¹, —CH₂OR¹¹, —OCH₂CO₂R¹¹, —CH₂SR¹¹, —CH₂N(R¹¹)(R¹²), —SR¹¹, —OSR¹¹, —N(R¹¹)SO₂R¹², —OR¹³, or triethylsiloxy,
where R¹³ is H, —C(O)—O—R¹⁴, or —C(O)(R¹⁴), where R¹⁴ is alkyl, aryl, or amino, and where R¹¹ and R¹² are each, independently, hydrogen, substituted or unsubstituted (C₁-C₅) alkyl, (C₂-C₅) alkenyl, (C₂-C₅) alkynyl, or cycloalkyl or aryl group having 3 to 10 carbon atoms;
wherein R⁶ is H or —OR⁸,
where R⁸ is H, or —C(O)R⁹, where R⁹ is alkyl, aryl, or amino;
wherein R⁷ is —CH₃;
wherein R³ is O and R⁵ is selected from H, OH, —C(CH₃)—C(O)—O(CH₃), (C₁-C₁₀) alkyl, (C₂-C₁₀) alkenyl, (C₂-C₁₀) alkynyl, substituted or unsubstituted aryl, substituted or unsubstituted heteroaryl, heterocyclic, amino, amido, alkoxy, alkenyloxy, alkynyloxy, —O-aryl, —O-(alkyl)(C₁-C₁₀), (—NH-alkyl, —N(alkyl)₂, —NH₂, -alkyl-C(O)(OH), -alkyl-OH, -alkyl-(NH₂), halide, CX₃ where X is a halide, indole radical, -A-Ar, -A-Z-Ar, —SO₂—Ar, -A-NR¹⁰, —O-A-Ar, or —R¹⁰, where A, Z, Ar and R¹⁰ are defined as above, or
wherein R⁵ is O and R³ is selected from OH, —C(CH₃)—C(O)—O(CH₃), (C₁-C₁₀) alkyl, (C₂-C₁₀) alkenyl, (C₂-C₁₀) alkynyl, substituted or unsubstituted aryl, substituted or unsubstituted heteroaryl, heterocyclic, amino, amido, alkoxy, alkenyloxy, alkynyloxy, —O-aryl, —O—(C₂-C₁₀ alkyl) (C₁-C₁₀), (—NH-alkyl, —N(alkyl)₂, —NH₂, -alkyl-C(O)(OH), -alkyl-OH, -alkyl-(NH₂), halide, CX₃ where X is a halide, indole radical, -A-Ar, -A-Z-Ar, —SO₂—Ar, -A-NR¹⁰, —O-A-Ar, or —R¹⁰, where A, Z, Ar and R¹⁰ are defined as above,
or an optically pure enantiomer, diastereomer, tautomer or salt thereof.

2. The compound of claim 1, having the structure:

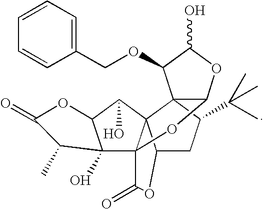

3. The compound of claim 1, having the structure:

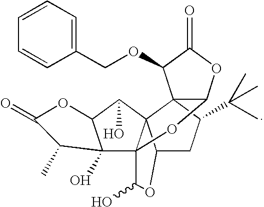

4. The compound of claim 1, wherein the compound has the structure:

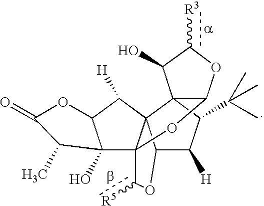

5. The compound of claim 1, wherein the compound has the structure:

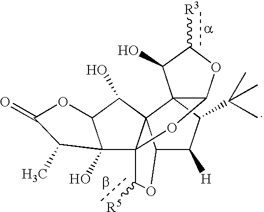

6. The compound of claim 1, wherein the compound has the structure:

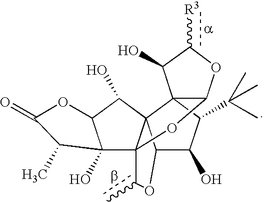

7. The compound of claim 1, wherein the compound has the structure:

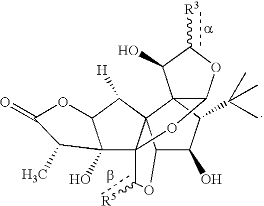

8. The compound of claim 1, wherein the compound has the structure:

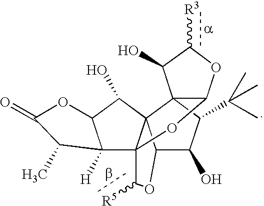

9. A process for preparing the compound of claim comprising:
(a) exposing a compound having the structure: 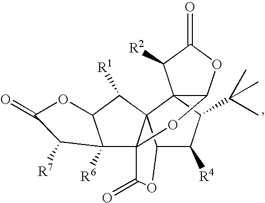
wherein R¹ is H or —OR⁸,
where R⁸ is H, or —C(O)R⁹, where R⁹ is alkyl, aryl, or amino;
wherein R² is H, OH, —O(CH₃), —OC(O)CH₃, (C₁-C₁₀) alkyl, (C₂-C₁₀) alkenyl, (C₂-C₁₀) alkynyl, -A-Ar, -A-Z-Ar, —SO₂—Ar, -A-NR¹⁰, O-A-Ar, or —R¹⁰,
where A is (C₁-C₈) alkyl, (C₂-C₈) alkenyl, (C₂-C₈) alkynyl, which is unsubstituted or substituted by a straight or branched alkyl chain group having 1 to 5 carbon atoms; Z is carbon, oxygen, sulfur or nitrogen; Ar is a phenyl group, a pyridyl group, a naphthyl group, a pyrimidyl group, or a quinolyl group, each of which is unsubstituted or substituted by one to five substituents selected from the group consisting of hydrogen, halogen, a hydroxy group, a carboxylic acid group, substituted or unsubstituted (C₁-C₁₀) alkyl, (C₂-C₁₀) alkenyl, (C₂-C₁₀) alkynyl, (C₁-C₁₀) haloalkyl, (C₁-C₁₀) alkoxy, (C₂-C₁₀) alkenyloxy, (C₂-C₁₀) alkynyloxy, (C₁-C₁₀) haloalkoxy, a phenyl group, a phenoxy group, an aralkyl group, an aralkyloxy group, a substituted phenyl group, a substituted phenoxy group, a substituted aralkyl group, a substituted aralkyloxy group, —C(O)R¹⁰, —C(O)NR¹⁰R¹⁰, —C(O)OR¹⁰, —N(H)COR¹⁰—NH(OH), —N(OH)COR¹⁰, —CH₂OR¹⁰, —OCH₂CO₂R¹⁰, —CH₂CO₂R¹⁰, —CH₂SR¹⁰, —CH₂NR¹⁰R¹⁰, —CH₂CONR¹⁰R¹⁰, —SR¹⁰, —OSR¹⁰, —N(R¹⁰)(R¹⁰), or —NR¹⁰SO₂R¹⁰,
where each R¹⁰ is independently selected from hydrogen, (C₁-C₁₀) alkyl, (C₃-C₁₀) cycloalkyl, —SCX₃ in which X is a halogen, —CN, —NO₂ or -Z-A-Z′- in which Z and A are as defined above and Z′ represents carbon, oxygen, sulfur, or nitrogen;
wherein R⁴ is H, OH, halide, unsubstituted or substituted, straight or branched (C₁-C₅) alkyl group, (C₂-C₅) alkenyl, or a (C₂-C₅) alkynyl, (C₁-C₅) alkoxy, (C₂-C₅) alkenyloxy, or (C₂-C₅) alkynyloxy, —N₃, —C(O)R¹¹, —C(O)NR¹¹R¹², —C(O)OR¹¹, —OC(O)R¹¹, —OC(O)OR¹¹, —NH(OH), —N(R¹¹)(R¹²), —N(H)CR¹¹, —N(OH)COR¹¹, —CH₂OR¹¹, —OCH₂CO₂R¹¹, —CH₂SR¹¹, —CH₂N(R¹¹)(R¹²), —SR¹¹, —OSR¹¹, —N(R¹¹)SO₂R¹², —OR¹³ or triethylsiloxy,
where R¹³ is H, —C(O)—O—R¹⁴, or —C(O)(R¹⁴), where R¹⁴ is alkyl, aryl, or amino, and where R¹¹ and R¹² are each, independently, hydrogen, substituted or unsubstituted (C₁-C₅) alkyl, (C₂-C₅) alkenyl, or (C₂-C₅) alkynyl, or a cycloalkyl or aryl group having 3 to 10 carbon atoms;
wherein R⁶ is H or —OR⁸,
where R⁸ is H, or —C(O)R⁹, where R⁹ is alkyl, aryl, or amino; and
wherein R⁷ is —CH₃,
to NaBH₄ in a suitable solvent to produce a lactol derivative; and
(b) reacting the lactol derivative product of step (a) with an agent suitable to produce the compound.

10. The process of claim 9, wherein the suitable solvent in step (a) is MeOH.

11. (canceled)

12. The process of claim 9, wherein the suitable agent is a carboxcylic acid, an alkylating reagent or an acid halide.

13. A process for preparing the compound of claim 1 comprising:
(a) exposing a compound having the structure: 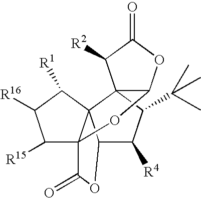
wherein R¹ is H or —OR⁸,
wherein R² is H, OH, —O(CH₃), —OC(O)CH₃, (C₁-C₁₀) alkyl, (C₂-C₁₀) alkenyl, (C₂-C₁₀) alkynyl, -A-Ar, -A-Z-Ar, —SO₂—Ar, -A-NR¹⁰, —O-A-Ar, or —R¹⁰,
where A is (C₁-C₈) alkyl, (C₂-C₈) alkenyl, (C₂-C₈) alkynyl, which is unsubstituted or substituted by a straight or branched alkyl chain group having 1 to 5 carbon atoms; Z is carbon, oxygen, sulfur or nitrogen; Ar is a phenyl group, a pyridyl group, a naphthyl group, a pyrimidyl group, or a quinolyl group, each of which is unsubstituted or substituted by one to five substituents selected from the group consisting of hydrogen, halogen, a hydroxy group, a carboxylic acid group, substituted or unsubstituted (C₁-C₁₀) alkyl, (C₂-C₁₀) alkenyl, (C₂-C₁₀) alkynyl, (C₁-C₁₀) haloalkyl, (C₁-C₁₀) alkoxy, (C₂-C₁₀) alkenyloxy, (C₂-C₁₀) alkynyloxy, (C₁-C₁₀) haloalkoxy, a phenyl group, a phenoxy group, an aralkyl group, an aralkyloxy group, a substituted phenyl group, a substituted phenoxy group, a substituted aralkyl group, a substituted aralkyloxy group, —C(O)R¹⁰, —C(O)NR¹⁰R¹⁰, —C(O)OR¹⁰, —N(H)COR¹⁰—NH(OH), —N(OH)COR¹⁰, —CH₂OR¹⁰, —OCH₂CO₂R¹⁰, —CH₂CO₂R¹⁰, —CH₂SR¹⁰, —CH₂NR¹⁰R¹⁰, —CH₂CONR¹⁰R¹⁰, —SR¹⁰—OSR¹⁰N(R¹⁰)(R¹⁰), or —NR¹⁰SO₂R¹⁰,
where each R¹⁰ is independently selected from hydrogen, (C₁-C₁₀) alkyl, (C₃-C₁₀) cycloalkyl, —SCX₃ in which X is a halogen, —CN, —NO₂ or -Z-A-Z′- in which Z and A are as defined above and Z′ represents carbon, oxygen, sulfur, or nitrogen;
wherein R⁴ is H, OH, halide, unsubstituted or substituted, straight or branched (C₁-C₅) alkyl group, (C₂-C₅) alkenyl, or a (C₂-C₅) alkynyl, (C₁-C₅) alkoxy, (C₂-C₅) alkenyloxy, or (C₂-C₅) alkynyloxy, —N₃, —C(O)R¹¹, —C(O)NR¹¹R¹², —C(O)OR¹¹, —OC(O)R¹¹, —OC(O)OR¹¹, —NH(OH), —N(R¹¹)(R¹²), —N(H)COR¹¹, —N(OH)COR¹¹, —CH₂OR¹¹, —OCH₂CO₂R¹¹, —CH₂SR¹¹, —CH₂N(R¹¹)(R¹²), —SR¹¹, —OSR¹¹, —N(R¹¹)SO₂R¹², —OR¹³, or triethylsiloxy,
where R¹³ is H, —C(O)—O—R¹⁴, or —C(O)(R¹⁴), where R¹⁴ is alkyl, aryl, or amino, and where R¹¹ and R¹² are each, independently, hydrogen, substituted or unsubstituted (C₁-C₅) alkyl, (C₂-C₅) alkenyl, (C₂-C₅) alkynyl, or cycloalkyl or aryl group having 3 to 10 carbon atoms; and
wherein R¹⁵ is H or halide, and R¹⁶ is —C(CH₃)—C(O)—OCH₃,
to NaBH₄ in a suitable solvent so as to produce a second compound having the structure: 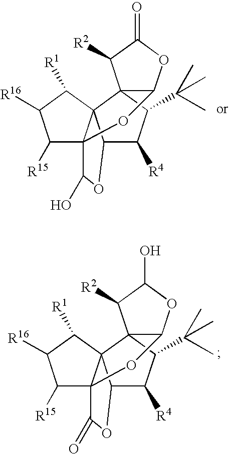
(b) reacting the lactol product of step (a) with an agent suitable to produce a compound having the structure: 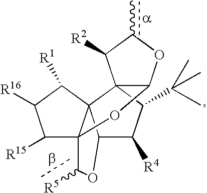
wherein R³ is O and R⁵ is selected from H, OH, —C(CH₃)—C(O)—O(CH₃), (C₁-C₁₀) alkyl, (C₂-C₁₀) alkenyl, (C₂-C₁₀) alkynyl, substituted or unsubstituted aryl, substituted or unsubstituted heteroaryl, heterocyclic, amino, amido, alkoxy, alkenyloxy, alkynyloxy, —O-aryl, —O-(alkyl)(C₁-C₁₀), (—NH-alkyl, —N(alkyl)₂, —NH₂, -alkyl-C(O)(OH), -alkyl-OH, -alkyl-(NH₂), halide, CX₃ where X is a halide, indole radical, -A-Ar, -A-Z-Ar, —SO₂—Ar, -A-NR¹⁰, —O-A-Ar, or —R¹⁰, where A, Z, Ar and R¹⁰ are defined as above, or
wherein R⁵ is O and R³ is selected from OH, —C(CH₃)—C(O)—O(CH₃), (C₁-C₁₀) alkyl, (C₂-C₁₀) alkenyl, (C₂-C₁₀) alkynyl, substituted or unsubstituted aryl, substituted or unsubstituted heteroaryl, heterocyclic, amino, amido, alkoxy, alkenyloxy, alkynyloxy, —O-aryl, —O—(C₂-C₁₀ alkyl) (C₁-C₁₀), (—NH-alkyl, —N(alkyl)₂, —NH₂, -alkyl-C(O) (OH), -alkyl-OH, -alkyl-(NH₂), halide, CX₃ where X is a halide, indole radical, -A-Ar, -A-Z-Ar, —SO₂—Ar, -A-NR¹⁰, —O-A-Ar, or —R¹⁰, where A, Z, Ar and R¹⁰ are defined as above; and
(c) joining R¹⁶ and R¹⁵ to form a lactone.

14. The process of claim 13, wherein the suitable solvent in step (a) is MeOH.

15. (canceled)

16. The process of claim 13, further comprising the step of exposing the compound produced in step (a) to p-phenylbenzoic acid, EDC and DMAP so as to resolve the enantiomers before step (b).

17. (canceled)

18. A process for preparing a compound having the structure: 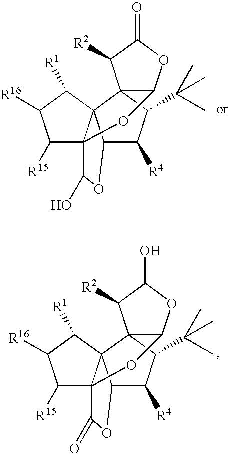
comprising reacting 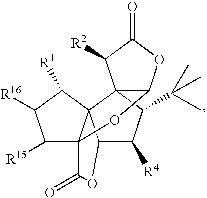
wherein R¹ is H or —OR⁸,
wherein R² is H, OH, —O(CH₃), —OC(O)CH₃, (C₁-C₁₀) alkyl, (C₂-C₁₀) alkenyl, (C₂-C₁₀) alkynyl, -A-Ar, -A-Z-Ar, —SO₂—Ar, -A-NR¹⁰, —O-A-Ar, or —R¹⁰,
where A is (C₁-C₈) alkyl, (C₂-C₈) alkenyl, (C₂-C₈) alkynyl, which is unsubstituted or substituted by a straight or branched alkyl chain group having 1 to 5 carbon atoms; Z is carbon, oxygen, sulfur or nitrogen; Ar is a phenyl group, a pyridyl group, a naphthyl group, a pyrimidyl group, or a quinolyl group, each of which is unsubstituted or substituted by one to five substituents selected from the group consisting of hydrogen, halogen, a hydroxy group, a carboxylic acid group, substituted or unsubstituted (C₁-C₁₀) alkyl, (C₂-C₁₀) alkenyl, (C₂-C₁₀) alkynyl, (C₁-C₁₀) haloalkyl, (C₁-C₁₀) alkoxy, (C₂-C₁₀) alkenyloxy, (C₂-C₁₀) alkynyloxy, (C₁-C₁₀) haloalkoxy, a phenyl group, a phenoxy group, an aralkyl group, an aralkyloxy group, a substituted phenyl group, a substituted phenoxy group, a substituted aralkyl group, a substituted aralkyloxy group, —C(O)R¹⁰, —C(O)NR¹⁰R¹⁰, —C(O)OR¹⁰, —N(H)COR¹⁰—NH(OH), —N(OH)COR¹⁰, —CH₂OR¹⁰, —OCH₂CO₂R¹⁰, —CH₂CO₂R¹⁰, —CH₂SR¹⁰, —CH₂NR¹⁰R¹⁰, —CH₂CONR¹⁰R¹⁰, —SR¹⁰, —OSR¹⁰, —N(R¹⁰)(R¹⁰), or —NR¹⁰SO₂R¹⁰,
where each R¹⁰ is independently selected from hydrogen, (C₁-C₁₀) alkyl, (C₃-C₁₀) cycloalkyl, —SCX₃ in which X is a halogen, —CN, —NO₂ or -Z-A-Z′- in which Z and A are as defined above and Z′ represents carbon, oxygen, sulfur, or nitrogen;
wherein R⁴ is H, OH, halide, unsubstituted or substituted, straight or branched (C₁-C₅) alkyl group, (C₂-C₅) alkenyl, or a (C₂-C₅) alkynyl, (C₁-C₅) alkoxy, (C₂-C₅) alkenyloxy, or (C₂-C₅) alkynyloxy, —N₃, —C(O)R¹¹, —C(O)NR¹¹R¹², —C(O)OR¹¹, —OC(O)R¹¹, —OC(O)OR¹¹, —NH(OH), —N(R¹¹)(R¹²), —N(H)COR¹¹, —N(OH)COR¹¹, —CH₂OR¹¹, —OCH₂CO₂R¹¹, —CH₂SR¹¹, —CH₂N(R¹¹)(R¹²), —SR¹¹, —OSR¹¹, —N(R¹¹)SO₂R¹², —OR¹³, or triethylsiloxy,
where R¹³ is H, —C(O)—O—R¹⁴, or —C(O)(R¹⁴), where R¹⁴ is alkyl, aryl, or amino, and where R¹¹ and R¹² are each, independently, hydrogen, substituted or unsubstituted (C₁-C₅) alkyl, (C₂-C₅) alkenyl, (C₂-C₅) alkynyl, or cycloalkyl or aryl group having 3 to 10 carbon atoms; and
wherein R¹⁵ is H or halide, and R¹⁶ is —C(CH₃)—C(O)—OCH₃,
with NaBH₄ in a suitable solvent.

19. The process of claim 18, wherein the compound produced is: 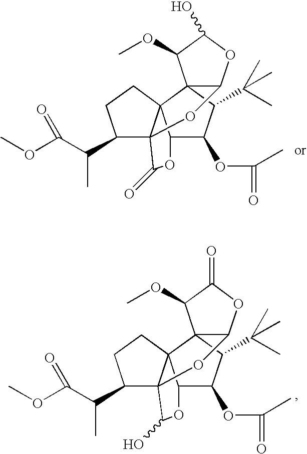
and the process comprises reacting: 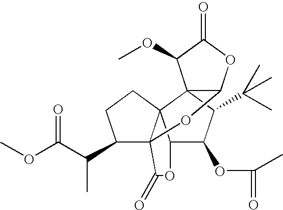
with NaBH₄ in a suitable solvent.

20. (canceled)

21. (canceled)

22. A process for preparing a compound having the structure: 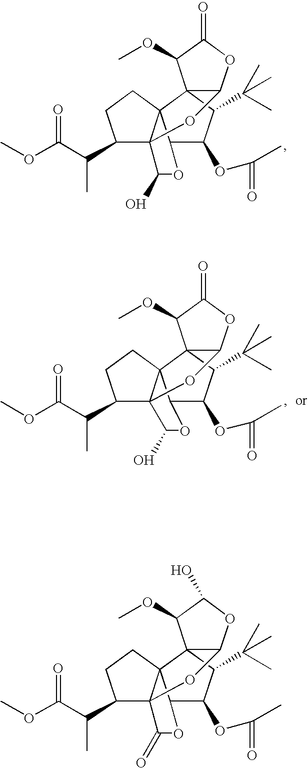
comprising:
a) exposing a compound having the structure: 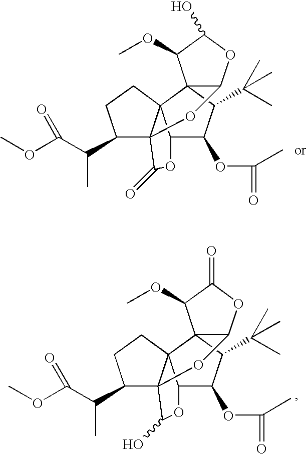
to p-phenylbenzoic acid, DEC, DMAP, at a suitable temperature so as to produce a compound having the structure: 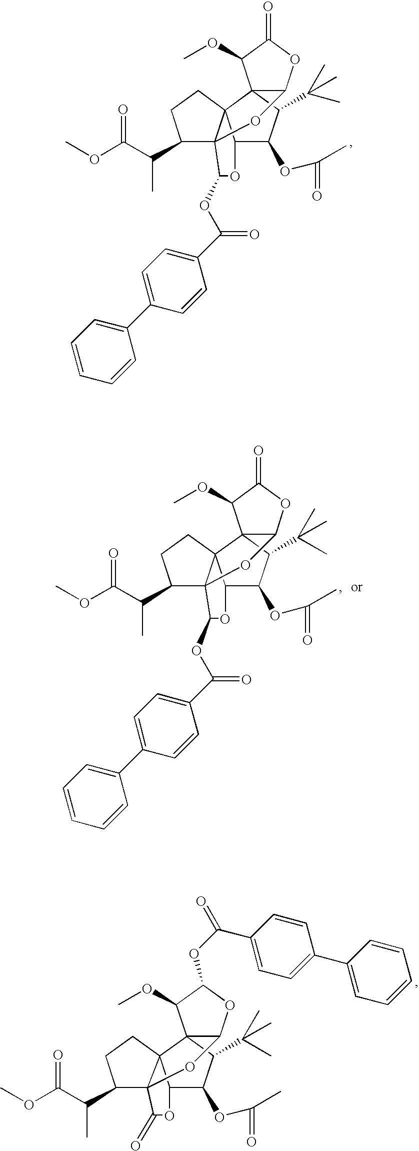
b) separating the compounds produced in step a); and
c) exposing the products of step b) to a suitable hydrolyzing agent so as to produce the compound.

23. The process of claim 22, wherein the hydrolyzing agent is K₂CO₃ in a suitable solvent.

24. (canceled)

25. (canceled)

26. A method of making a composition comprising admixing an effective amount of the compound of claim 1 and a pharmaceutically acceptable carrier.

27. A composition comprising the compound of claim 1 and a carrier.
